(19) United States
(12) Patent Application Publication
(10) Pub. No.: US 2008/0058856 A1
(43) Pub. Date: Mar. 6, 2008

(54) Non-occluding dilation device

(76) Inventors: Venkatesh Ramaiah, Scottsdale, AZ (US); Robert McNutt, Mesa, AZ (US)

Correspondence Address: SQUIRE SANDERS & DEMPSEY LLP, TWO RENAISSANCE SQUARE, 40 NORTH CENTRAL AVENUE, SUITE 2700, PHOENIX, AZ, 85004-4498, US

(21) Appl. No.: 11/820,726

(22) Filed: Jun. 19, 2007

Related U.S. Application Data
(63) Continuation-in-part of application No. 11/478,340, filed on Jun. 28, 2006.
(60) Provisional application No. 60/595,378, filed on Jun. 28, 2005.

(51) Int. Cl. A61M 29/00

(52) U.S. Cl. 606/198

(57) ABSTRACT

A device for dilating a vessel or a structure (such as a stent or stent graft) within a vessel comprises a plurality of wires that are spaced apart when the device is dilated so as to allow fluid to flow through the device. Thus, when in use the device does not occlude or substantially hinder the flow of blood through a vessel or into side vessels.

### CROSS REFERENCE TO RELATED APPLICATIONS

This application claims priority to U.S. Utility Application Ser. No. 11/478,340, filed Jun. 28, 2006, the contents of which are incorporated by reference herein.

### FIELD OF THE INVENTION

The present invention relates generally to medical devices, and more particularly to a medical device for the dilation of blood vessels and/or the dilation of structures positioned within blood vessels.

### BACKGROUND OF THE INVENTION

Conventional systems for dilating blood vessels and/or structures (e.g., stents or stent grafts) positioned in a blood vessel utilize balloon-like structures. Such structures are made from essentially impermeable materials. When such a device is expanded to perform the dilation, blood flow is occluded through the blood vessel in which the balloon-like dilator is being used. Such an occlusion of blood flow could, if continued for too long, harm the patient, since portions of the body will not receive blood while the flow is occluded or substantially hindered. Thus, the length of time balloon-like dilators may be dilated is limited and this can hinder proper completion of the dilation procedure.

A similar problem with balloon-like dilators arises when a dilation procedure is being performed in a portion of the circulatory system where there is a branch in the blood vessels, such as where the iliac or renal arteries branch from the aorta. For example, when the balloon-like dilator is used in the aorta it may cover a side vessel and partially or totally occlude blood flow to the side vessel.

Another problem with balloon-like dilators is called the “windsock effect.” Because blood flow is substantially or entirely occluded when balloon-like dilators are dilated, the blood pressure upstream of the dilator can be significant and may cause the balloon-like dilator, and any structure (such as a stent or stent graft) positioned in the blood vessel and that was being dilated, to move out of the desired position, effectively pushed down stream (i.e., in the antegrade direction) by the blood. As such, accurate placement of such structures can be difficult utilizing balloon dilators.

### DEFINITIONS

As used herein, in addition to the other terms defined in this disclosure, the following terms shall have the following meanings:

“Collapsed” when referring to a device according to the invention means that the device is in its relaxed, undilated position. The device would normally be in its collapsed position when introduced into a vessel.

“Criss-cross” pattern means a wire pattern wherein the wires cross one another as shown, for example, in FIGS. 13-20.

“Device” means a structure for (a) dilating a vessel and/or (b) dilating a structure inside of a vessel (such as an endograft stent or stent graft) to be deployed or repositioned within a vessel.

“Diameter” as used in connection with a vessel means the approximate diameter of a vessel since vessels are seldom perfectly cylindrical.

“Diameter disparity ratio” means the disparity of the diameter of a single vessel. Vessels, particularly diseased vessels, may not have a relatively constant diameter and the diameter can suddenly increase or decrease. For example, the diameter of a vessel may suddenly change from an initial diameter to a diameter of 1.5 times the initial diameter, in which case the diameter disparity ratio would be 1.5:1. A diameter disparity ratio or multi-vessel diameter disparity ratio (as defined below) to which a device according to some aspects of the invention could conform is one or more of the ratios between 1.2:1 and 3.0:1, including diameter disparity ratios of 1.2:1, 1.4:1, 1.6:1, 1.8:1, 2.0:1, 2.2:1, 2.4:1, 2.6:1 and 2.8:1 and 3.0:1. One device according to the invention can conform to a diameter disparity ratio of about 3.4:1 in a test simulating the operation of the device in a vessel.

“Dilated” refers to a device according to the invention when it is expanded. When dilated within a vessel a device according to the invention is expanded to conform to the approximate inner dimensions of the vessel. A device dilated within a vessel may be dilated for the purpose of dilating the vessel itself or for dilating a structure within the vessel. “Expanded” and “dilated” have the same meaning when used in connection with a device.

“Fluid” means any bodily fluid, such as blood.

“Fully dilated” means the maximum amount a device according to the invention can be dilated when included in a delivery catheter and dilated using the catheter's delivery system.

“Kink radius” refers to one-half the diameter at which a device according to the invention can be formed without the device permanently deforming (i.e., without “kinking”). The lower the kink radius the greater the resistance of the device to kinking. FIGS. 21-23 show measurement of the kink radius with respect to an embodiment of the present invention. “Kink radius” and methods for testing same are discussed in “Pigtail Catheters Used for Percutaneous Fluid Drainage: Comparison of Performance Characteristics,” Douglas B. Macha, John Thomas and Rendon C. Nelson, Radiology vol. 238: Number 3 (March 2006), the contents of which that are related to kink testing are incorporated herein by reference.

“Multi-vessel diameter disparity ratio” means the disparity of the diameters of two vessels. When a device according to the invention is used it may be deployed and dilated within two vessels simultaneously and the two vessels may have different, respective diameters. For example, if one vessel has a first diameter and the second vessel has a second diameter 1.8 times as large as the first diameter, the multi-vessel diameter disparity ratio would be 1.8:1. A device according to some aspects of the invention could conform to one or more of the multi-vessel diameter ratios between 1.2:1 and 3.0:1.

“Pressure drop” means the reduction in pressure in part of a vessel when a device is (a) dilated within the vessel, or (b) dilated in another vessel but totally or partially covering the opening to the vessel (in which case the vessel may be referred to as a “side vessel”). When a standard balloon device is fully dilated within a vessel the pressure upstream of the balloon device increases significantly while the pressure downstream of the balloon device, or in a side vessel covered by the balloon device, can reach substantially zero (meaning that the balloon has blocked most or all of the blood flow). As an example, if the pressure at a location in a vessel is 100 mm Hg before a device is dilated, and the pressure at the same location in the vessel is 10 mm HG after the device is dilated, the pressure drop would be 90%, i.e., 100−10=90, and 90/100=90%. Similarly, for the same vessel if the pressure after dilation were 20 mm Hg the pressure drop would be 80%, if the pressure after dilation were 30 mm Hg the pressure drop would be 70%, if the pressure after dilation were 5 mm Hg the pressure drop would be 95% and if the pressure after dilation were 1 mm Hg the pressure drop would be 99%.

“Strut” means a wire having a generally rectangular cross-section with generally flat surfaces and having a width greater than its thickness.

“Vessel” means any vessel within a body, such as the human body, through which blood or other fluid flows and includes arteries and veins.

“Vessel flow path” means the direction of fluid flow through a vessel.

“Wire” means any type of wire, strand, strut or structure, regardless of cross-sectional dimension (e.g., the cross-section could be circular, oval, or rectangular) or shape, and regardless of material, that may be used to construct any of the devices as described or claimed herein. Some wires may be suitable for one or more of the embodiments but not suitable for others.

### SUMMARY OF THE INVENTION

The present invention provides a device for dilating either a vessel or a structure positioned within the vessel. The device may be used in any medical application in which dilation of a vessel or dilation of a structure positioned within a vessel (e.g., a stent or stent graft, such as a thoracic or abdominal aortic stent graft) is desired. The device is designed so that when it is expanded it does not occlude or substantially hinder the flow of fluid through the vessel or through side vessels that connect to the vessel. The device includes a plurality of wires and has a first position in which the device is not dilated and can be moved into or retrieved from the vessel, and a second position in which the device is expanded and dilates the vessel and/or a structure. When dilated, fluid passes through the openings between the wires rather than being occluded or substantially hindered.

According to one embodiment of the invention, the device comprises a wire mesh that may be spiraled, formed in a criss-cross pattern or formed in any suitable pattern. The device can then be contracted for removal from the vessel. The expansion and contraction of the device may be accomplished using a twisting motion or by applying linear pressure to the device such as through a pushing or pulling motion by an operator, which compresses it and causes the device to dilate. The device can be collapsed by reversing the twisting motion or by releasing the linear pressure.

According to another embodiment of the invention, the device comprises a plurality of wires that are substantially parallel to the vessel flow path when inserted in a vessel. The expansion and contraction of such a device is preferably accomplished by applying linear pressure to the device such as through a pushing or pulling motion by an operator to compress the device and expand it, and by releasing the linear pressure to collapse the device.

Any device according to the invention may be preshaped so that it automatically expands into position when released from a catheter sheath. It can then be dilated further or contracted by an operator. An additional advantage of this particular design is that it takes less time and operator effort to dilate or contract the device to the proper dimension for use in a procedure.

Any device according to the invention is preferably mounted on a catheter and, utilizing the catheter, the device is positioned at the proper place within a vessel and then dilated.

The descriptions of the invention herein are exemplary and explanatory only and are not restrictive of the invention as claimed.

### BRIEF DESCRIPTION OF THE DRAWINGS

FIGS. 1A-E show examples of dilation devices according to various aspects of the invention.

FIGS. 2A-C show a spiraled dilation device according to one embodiment of the invention.

FIGS. 3A-D show additional views of a spiraled dilation device according to one embodiment of the invention.

FIGS. 4A-C show a non-spiraled, dilation device according to one embodiment of the invention.

FIGS. 5A-B show another non-spiraled, dilation device according to one embodiment of the invention.

FIGS. 6A-B show a delivery and deployment system for a non-spiraled, dilation device according to one embodiment of the invention.

FIG. 7 shows a control mechanism for a dilation device according to one embodiment of the invention.

FIG. 8 is a side view of an alternate device according to the invention in a dilated position and showing a band 808 in its body.

FIG. 9 shows a side view of an alternate device according to the invention in a dilated position and having wires that are parallel to the vessel flow path when the device is positioned in a vessel.

FIG. 10 shows a side view of an alternate device according to the invention in a dilated position and having wires that are parallel to the vessel flow path when the device is positioned in a vessel.

FIG. 11 shows a side view of an alternate device according to the invention in a dilated position and having wires that are parallel to the vessel flow path when the device is positioned in a vessel.

FIG. 12 is another side view of the device of FIG. 11.

FIG. 13 is a side view of an alternate embodiment of a device according to the invention and mounted on a catheter, wherein the device comprises wires formed in a criss-cross pattern.

FIG. 14 is a close up, partial side view of the device shown in FIG. 13.

FIG. 15 is another view of the device and catheter shown in FIG. 13 illustrating how the device can be used to dilate a stent graft.

FIG. 16 is a partial, side view of an alternate device according to the invention simulating how the device conforms to a diameter disparity ratio within a vessel.

FIG. 17 is a view of the device and catheter of FIG. 13 simulating how the device conforms to a multi-vessel diameter disparity ratio.

FIG. 18 is a view of the device of FIG. 16 simulating how the device conforms to a multi-vessel diameter disparity ratio and simultaneously conforms to an asymmetrical vessel shape.

FIG. 19 is another view of the device of FIG. 16 simulating how the device conforms to a diameter disparity ratio within a vessel and showing the device covering side vessels.

FIG. 20 is another view of the device of FIG. 13 simulating the device placed in the aorta and covering the renal arteries.

FIG. 21 is another view of the device of FIG. 13 showing that it has a kink radius of at least 13.5 mm when collapsed.

FIG. 22 is another view of the device of FIG. 13 showing that it has a kink radius of at least 16 mm when fully dilated.

FIG. 23 is another view of the device of FIG. 13 showing that it has a kink radius of at least 20 mm when fully dilated.

FIG. 24 is a side, perspective view of the catheter and device of FIG. 13.

FIG. 25 is a top view of the proximal end of the catheter of FIG. 13 with the device enclosed within the catheter's outer sheath.

FIG. 26 is a top view of the proximal end of the catheter of FIG. 13.

### DETAILED DESCRIPTION OF PREFERRED EMBODIMENTS

A device according to the invention is for dilating a vessel or a structure (such as an endograft, stent or stent graft) positioned in the vessel, or alternatively may be used to simultaneously dilate two vessels or to dilate a structure positioned in two vessels. The device comprises a plurality of wires and has a first position wherein it is collapsed. In this first position the device has a sufficiently small enough diameter to be positioned in a vessel where it is to be used. The device also has a second position wherein it is dilated in order to dilate either a vessel or a structure within the vessel. When dilated the wires are spaced apart to allow for the passage of fluid through the device. Thus, the device is designed so that it does not occlude or substantially hinder the flow of fluid through the vessel, so that when dilated for up to one minute there is little or no risk of necrosis due to lack of blood flow.

Some devices according to the invention are also sufficiently compliant (flexible) so that when placed in a vessel and dilated they conform to the dimensions of the vessel even when the dimensions are not uniform. In particular, the devices of the present invention are able to conform to vessels having one or more diameter disparity ratios of between 1.2:1 and 3.0:1. Some devices according to the invention can conform to one or more multi-vessel diameter disparity ratios of between 1.2:1 and 3.0:1.

The wires used in a device according to the invention may be of any suitable size, shape or material. For example, all or some of the wires may have a circular cross-section and have a diameter of between 0.008″ and 0.012″. Alternatively, all or some of the wires may include one or more slats that have a generally rectangular cross section and a thickness of between 0.008″ and 0.015″ and a width of between 0.020″ and 0.050″. The wire may be comprised of stainless steel, nitinol, cobalt, chromium or any suitable metal, plastic or other material. In a preferred embodiment, the wire is comprised of nitinol.

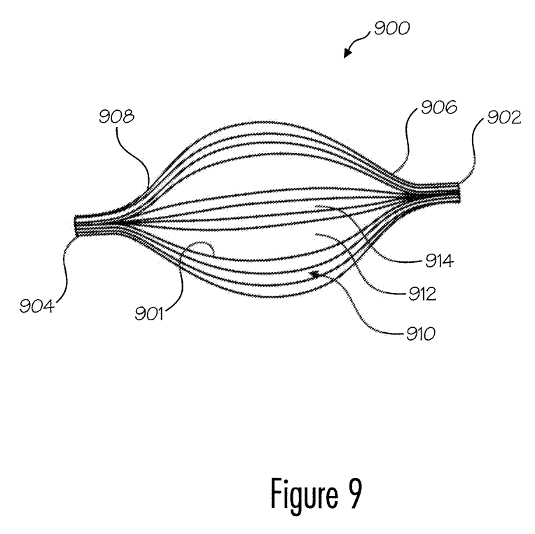

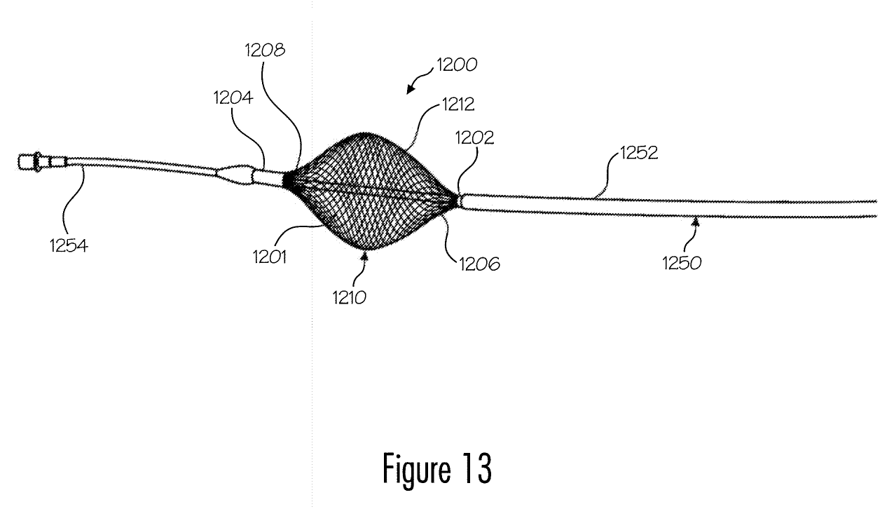

The device may have any suitable density of wires and the wires may be formed in any suitable pattern, such as in a criss-cross pattern (as shown in FIGS. 13-20) or in a non-overlapping pattern in which the wires are parallel to vessel flow path (as shown in FIGS. 9-12).

If a device according to the invention has wires that are parallel (as used in this context, “parallel” means substantially parallel) to the vessel flow path, the device may have between four and twenty-four wires, or may have more than twenty-four wires. In various embodiments, a device according to the invention includes, respectively, four wires, five wires, six wires, seven wires, eight wires, nine wires, ten wires, eleven wires, twelve wires, thirteen wires, fourteen wires, fifteen wires, sixteen wires, seventeen wires, eighteen wires, nineteen wires, twenty wires, twenty-one wires, twenty-two wires, twenty-three wires and twenty-four wires. The maximum distance between each wire in such a device can vary depending upon the number of wires, the width of the wires and the proposed use of the device, but generally the maximum distance between wires will be between 1 mm and 100 mm when the device is fully dilated. In various embodiments of the device, the maximum distance is, respectively, no greater than 1 mm, 5 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm and 100 mm.

If a device according to the invention includes wires in a criss-cross pattern, each of the largest spaces between the wires when the device is fully dilated could have an area of between 1 mm² and 400 mm², including areas of 1 mm², 2 mm², 4 mm², 10 mm², 25 mm², 50 mm², 75 mm², 100 mm², 150 mm², 200 mm², 250 mm², 300 mm², 350 mm², and/or 400 mm² or areas within that range. It is also possible that the area of the largest spaces could be larger than 400 mm² or smaller than 1 mm², as long as the device falls within the scope of one of the claims and works for its intended purpose of dilating a vessel or dilating a structure within a vessel without occluding or substantially hindering fluid flow through the vessel.

A device according to the invention may also have spaces between the wires that are greater in the central portion of the device than at the ends of the device, as illustrated, for example, in FIGS. 9-15 and 17-20.

A device according to the invention may be constructed in any suitable size or manner to accommodate a particular vessel, including veins and arteries (e.g., the abdominal aorta, aortic arch, the ascending aorta, the descending aorta, an iliac artery, or a renal artery). For example, the device may be used in wall apposition of a thoracic and/or abdominal endoluminal grafts, which means it expands to position at which at least a portion of the graft is snugly pressed against the artery wall. The dilation device may be introduced into a vessel either biaxially or triaxially (i.e., with a sheath or without) utilizing a catheter that is typically inserted over a guide wire. Optionally, the dilation device includes one or more radio opaque markers that assist an operator in locating the device once in a vessel although a device according to the invention can generally be seen using fluoroscopy without the need for radio opaque markers.

A device according to the invention may be dilated and contracted using any suitable method or structure, such as by applying and releasing linear pressure or by twisting and untwisting the device.

When dilated, devices according to the invention do not occlude or substantially hinder the flow of fluid through a vessel or into a side vessel because the fluids flow through the spaces (or openings) between the wires. In a pressure monitoring test using water as the fluid and a plastic tube to simulate the aorta the pressure drop within a vessel and downstream of a dilated device as generally shown in FIGS. 13-20 was measured as less than 1%. This test measured the flow lengthwise through the device, wherein the water had to flow through both the proximal end and distal end. Thus, the water had to flow through the smallest openings in the device. It is therefore believed that flow into a side vessel, wherein fluid would flow through the smaller openings in the distal end of the device and then through the larger openings in the body portion and into the side vessel, would be less hindered than flow lengthwise through the device. It is therefore believed that the pressure drop in a side vessel covered by a dilated device according to the invention (such as when the device is in the aorta and covers one or both renal arteries) would be less than 70%, less than 60%, less than 50%, less than 40%, less than 30%, less than 20%, less than 10%, less than 5%, less than 2% and/or less than 1%.

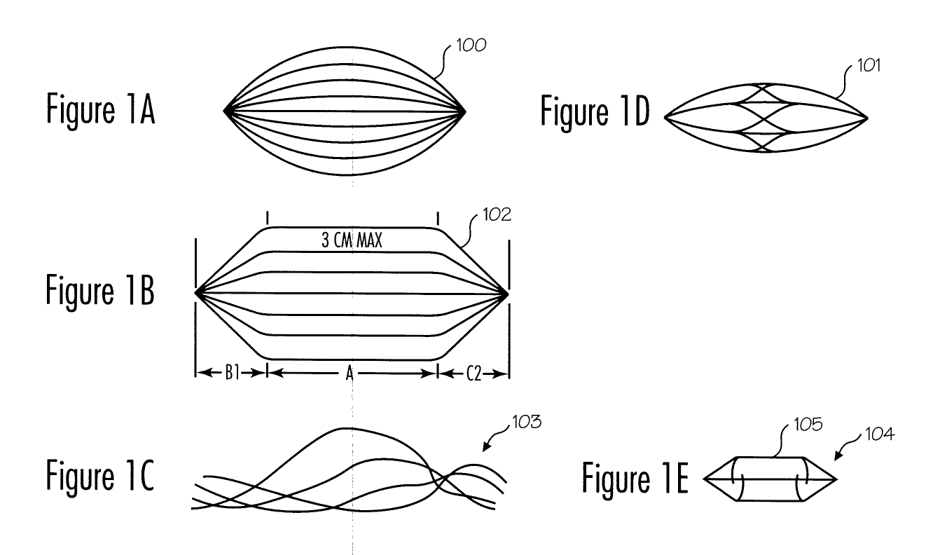

Reference will now be made to preferred embodiments of the invention, examples of which are illustrated in the accompanying drawings, wherein the purpose is to describe certain examples of the invention and not to limit the scope of the claims. FIGS. 1A-E show examples of spiraled devices according to various aspects of the invention. These devices are preferably dilated and collapsed by winding (to collapse) and unwinding (to dilate) a plurality of wires that are preferably formed in a spiraled pattern. Device 100 shown in FIG. 1A is a generally oval-shaped dilation device in a dilated position. Device 102 shown in FIG. 1B is a dilation device with a substantially-linear section A of wires in the middle of device 102, while the wires in end sections B1 and B2 are bent at an angle so that they converge at approximately the same point at each respective end 102A, 102B on either side of device 102. In this way, section A of dilation device 102 may exert more even pressure against a vessel and/or structure within a vessel (which would be positioned along section A during dilation). In this example, the substantially-straight section A is approximately 3 cm in length, while each of the end sections B1 and B2 is approximately 1 cm in length. However, the device may be of any suitable size or shape and be constructed in any manner. Device 101 shown in FIG. 1D, is a spiraled device in dilated position and includes support members 101A between wires 101B. Support members 101A provide additional strength to device 101.

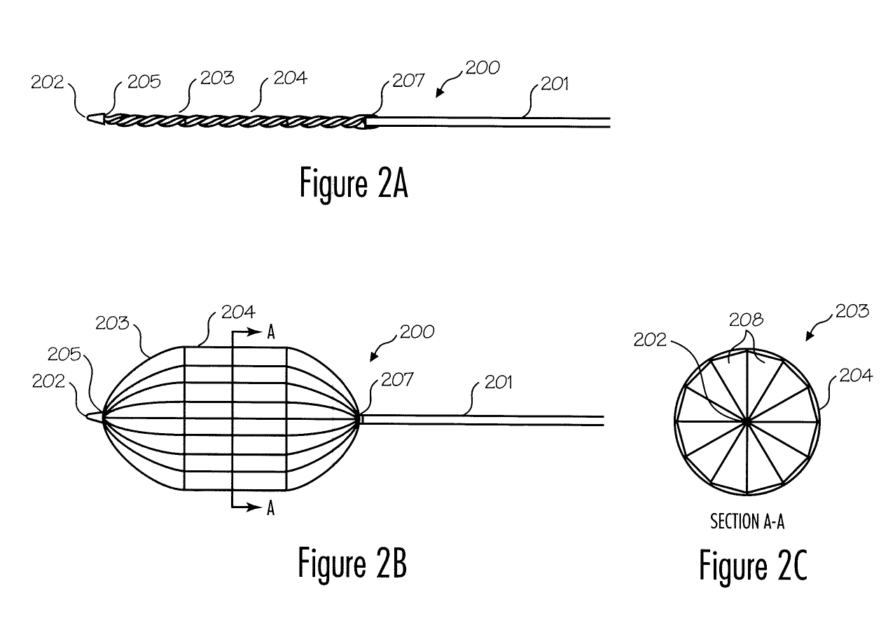

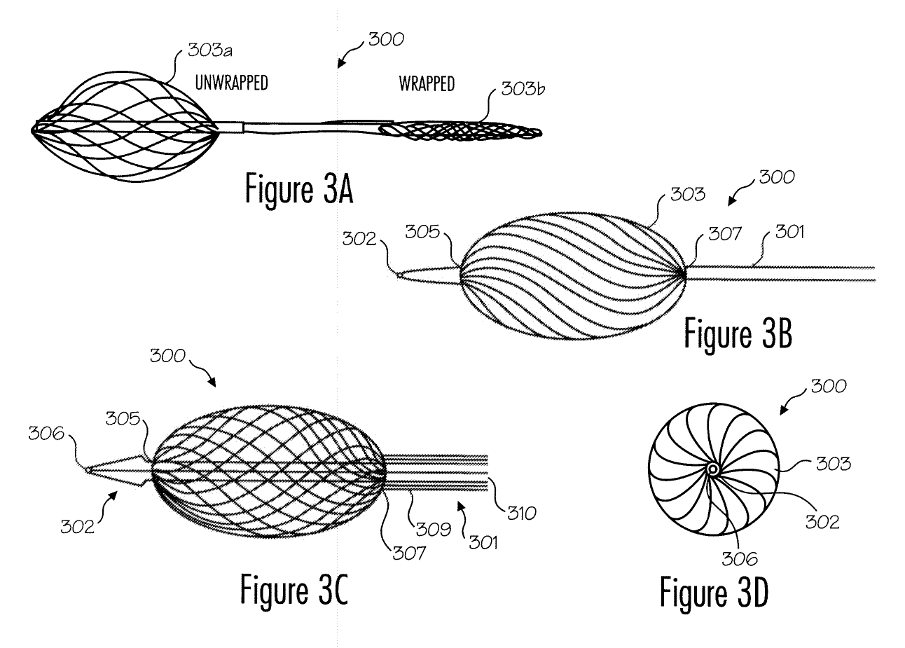

Device 103 shown in FIG. 1C is an exaggerated view of wires in a spiraled dilation device such as device 100 when the wires are in a spiraled position. In this position, the diameter of the dilation device is reduced, allowing for insertion into a blood vessel. Unspiraling the wires causes the device to dilate, as shown in devices 100, 101, and 102. Other embodiments of a spiraled dilation device will be discussed further with regard to FIGS. 2A-C and FIGS. 3A-D.

Any dilation device according to the invention may utilize a lining, such as lining 105 shown in FIG. 1E. A lining such as lining 105 may be positioned on part of the exterior surface and/or interior surface of device 104, or of any device according to the invention. The use of a lining (1) provides a more even surface (depending upon the nature of the device with which it is used) for exerting pressure during the dilation process, and/or (2) helps to prevent the wires of the device from becoming entangled with exposed wires on a stent or stent graft.

Lining 105 is preferably made from a permeable material, which would be important if the lining is positioned such that it could occlude or seriously hinder blood flow. However, impermeable materials may used if the lining is not positioned where it could seriously hinder blood flow. For example, in device 104, even if an impermeable material is used for the liner, blood will still flow through the gaps between the wires at each end of the device. So as long as device 104 is not positioned so that it blocks a side vessel, an impermeable membrane could optionally be used. Examples of preferable lining materials include, but are not limited to, polyurethane, PTFE (polytetrafluoroethylene), nylon, or any material used in carotid embolic protection devices. However, any material suitable for use inside vessels may be used.

FIGS. 2A-C show a spiraled dilation device according to one embodiment of the invention. FIG. 2A shows a spiraled dilation device 200 in a first position for insertion into a blood vessel. Device 200 includes a catheter 201 with a distal tip 202. Catheter 201 may be made of any material suitable for insertion into a blood vessel and capable of supporting a central lumen. Catheter 201 has a central lumen running the length of the catheter to a wire port (not shown) in distal tip 202. Catheter 201 is inserted into a blood vessel over a guide wire going through the wire port in distal tip 202 and through the central lumen. Catheter 201 may be any device having a central lumen and being capable of insertion into a blood vessel over a guide wire. Catheter 201 may be constructed in varying sizes to accommodate different sized vessels.

Dilation device 203 is affixed to catheter 201 near distal tip 202 at point 205 and at point 207. As shown in FIG. 2A, dilation device 203 is spiraled around the catheter is a first position. In this position, the catheter and dilation device are insertable into the blood vessel. Dilation device 203 may optionally include a lining 204 as discussed above with reference to FIG. 1.

FIG. 2B shows device 200 in an expanded (or dilated) position. Dilation device 203 is expanded by exerting a twisting motion on catheter 201. Because dilation device 203 is affixed at point 205 and at point 207, a twisting motion applied to catheter 201 will unspiral the device. The operation of the unspiraling mechanism will be discussed in more detail with reference to FIG. 3C. As can be seen in FIG. 2B, the use of optional lining 204 creates a substantially uniform surface for dilating blood vessels and structures.

FIG. 2C shows a top view of section A-A when then dilation device 203 is in the expanded position. As can be seen in the top view, lining 204 provides for a more substantially uniform surface for dilating than would the wire mesh of dilation device 203 alone. Gaps 208 between the wires of dilation device 203 allow blood, and other fluids to flow through the device and down the blood vessel.

FIGS. 3A-D show additional views of a spiraled dilation device according to one embodiment of the invention. FIG. 3A shows a spiral mesh structure rather than the straighter, cage-like structure of FIGS. 2A-C. In addition, the spiral mesh shown in FIG. 3A is denser than the structure shown in FIGS. 2A-C. The density of wires (i.e., the number of wires per area) used in the dilation devices may be varied for different applications. In general, the denser the wire when a device is dilated mesh, the more surface area available to dilate a blood vessel or device within the blood vessel. Dilation device 303a is shown in its expanded (or dilated) position, while dilation device 303b is shown in its non-expanded position. FIG. 3B shows an expanded spiral mesh device, including catheter 301, dilation device 303, affixation points 305 and 307, and distal tip 302.

FIG. 3C shows device 300 in more detail. Distal tip 302 as shown has a tapered front end. While not necessary, a tapered front end allows for easier insertion into a blood vessel if used biaxially or, if an additional sheath if used, triaxially. At the end of distal tip 302 is a wire port 306 for insertion over a guide wire 310. The proximal end of distal tip 302 may have a reverse taper to affixation point 305. Affixation point 305 is the point at which the distal end of dilation device 303 connects to distal tip 302 of catheter 301. Affixation point 307 is the point at which the proximal end of dilation device 303 connects to secondary sheath 309. Secondary sheath 309 is positioned coaxially around catheter 301. Dilation device 303 is expanded by twisting secondary sheath 309. This is accomplished because the portion of dilation device 303 attached to secondary sheath 309 at affixation point 307 moves (i.e., twists), while the portion of dilation device 303 attached to distal tip 302 of catheter 301 at affixation point 305 remains stationary. As such, dilation device 303 unspirals (or unwraps) when secondary sheath 309 is twisted. FIG. 3D shows a top view of device 300.

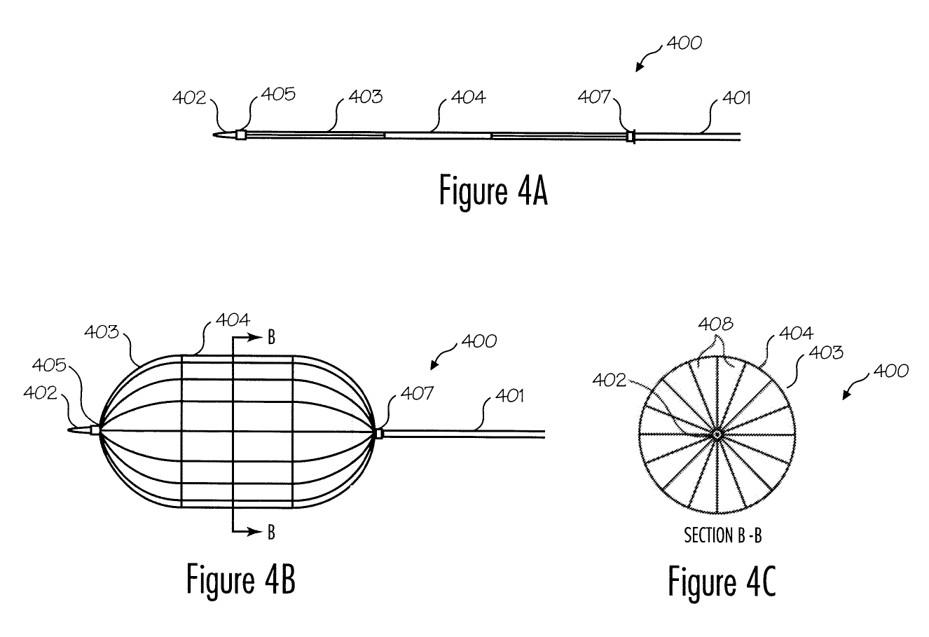

FIGS. 4A-C show a non-spiraled, expansive dilation device according to one embodiment of the invention. FIG. 4A shows a non-spiraled, expansive dilation device 400 in a first position for insertion into a blood vessel. Device 400 includes a catheter 401 with a distal tip 402. Catheter 401 may be any device having a central lumen and being capable of insertion into a blood vessel over a guide wire. Catheter 401 may be constructed in varying sizes to accommodate different blood vessels. Catheter 401 may be made of any material suitable for insertion into a blood vessel and capable of supporting a central lumen. Catheter 401 has a central lumen running the length of the catheter to a wire port (not shown) in distal tip 402. Catheter 401 is inserted into a blood vessel over a guide wire going through the wire port in distal tip 402 and through the central lumen.

Dilation device 403 is affixed to catheter 401 near distal tip 402 at point 405 and at point 407. As shown in FIG. 4A, dilation device 403 is not spiraled, but rather is affixed in a linear fashion in the first position. That is, each wire of dilation device 403 runs in a substantially straight line from affixation point 405 to affixation point 407. In this first position, the catheter and dilation device are insertable into the blood vessel. Dilation device 403 may optionally include a lining 404 as discussed above with reference to FIG. 1, which, as shown in FIGS. 4A-4C, is on the inside of dilation device 403.

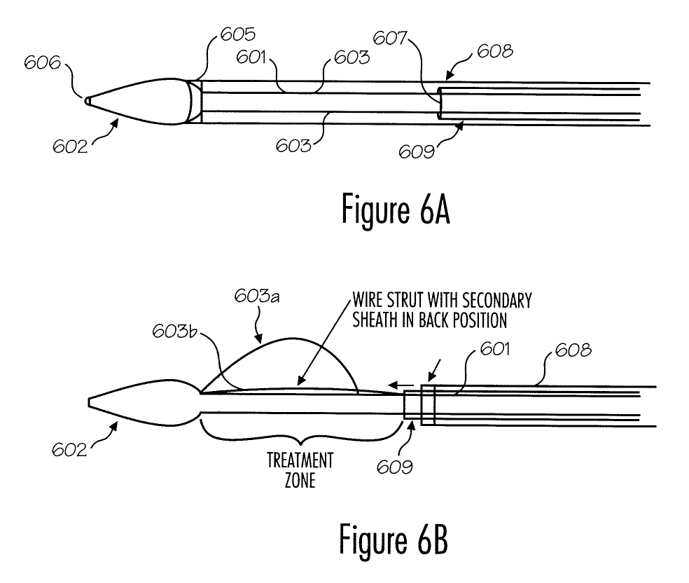

FIG. 4B shows device 400 in an expanded position. Dilation device 403 is expanded by exerting linear pressure on catheter 401 (e.g., a push-pull motion). Because dilation device 403 is affixed at points 405 and 407, a linear motion applied to catheter 401 will expand the device. The linear deployment mechanism will be discussed in more detail with reference to FIG. 6. As can be seen in FIG. 4B, the use of optional lining 404 creates a substantially uniform surface for dilating blood vessels and structures.

FIGS. 4C shows a top view of section A-A when then dilation device 403 is in the expanded position. As can be seen in the top view, lining 404 provides for a more substantially uniform surface for dilating. Gaps 408 between the wires of dilation device 403 allow blood, medicine, and other bodily fluids to flow through the device and down the vessel.

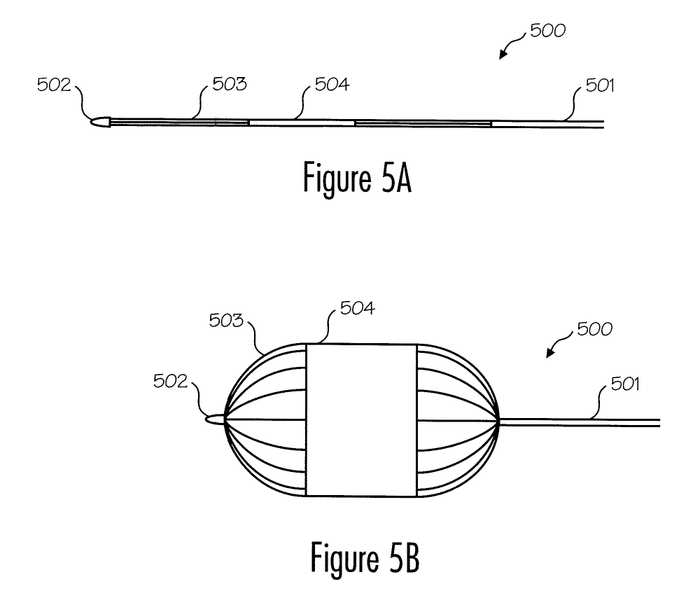

FIG. 5 shows another non-spiraled, expansive dilation device according to one embodiment of the invention. Device 500 includes a catheter 501, a distal tip 502 and is the same as device 400 except that liner 504 is placed on the outside of dilation device 503.

FIGS. 6A-B show a delivery and deployment system for a non-spiraled, expansive dilation device according to an embodiment of the invention. Catheter 601 includes a distal tip 602 with a wire port 606. Wire port 606 may be constructed to fit over any size guide wire (e.g., may be 0.038″ wire port). Again, distal tip 602 may be tapered at the tip for easier insertion into a blood vessel or addition sheath. Distal tip 602 may also be reversed tapered to affixation point 605. Affixation point 605 is where the distal end of dilation device 603 attaches to catheter 601. Secondary sheath 609 is positioned coaxially around catheter 601. The proximal end of dilation device 603 attaches to secondary sheath 609 at affixation point 607. An additional outer sheath 608 is positioned coaxially around catheter 601 and secondary sheath 609.

FIG. 6B shows the non-spiraled, expansive dilation device in two positions. In position 603a, dilation device 603 is expanded. The expansion is accomplished by pushing or screwing secondary sheath 609 forward. In this way, the proximal end of dilation device 603 is pushed forward while the distal end of dilation device 603 remains stationary because it is affixed to distal tip 602 of catheter 601. As such, the wires of dilation device 603 are pushed forward and expand to a predetermined maximum diameter. In position 603b, the wires of dilation device 603 remain at their smallest diameter and close to the catheter 601. This position is achieved by pulling secondary sheath 609 back until distal tip 602 butts against outer sheath 608. Outer sheath 608 may include radiopaque markers to indicate when device has cleared the treatment zone.

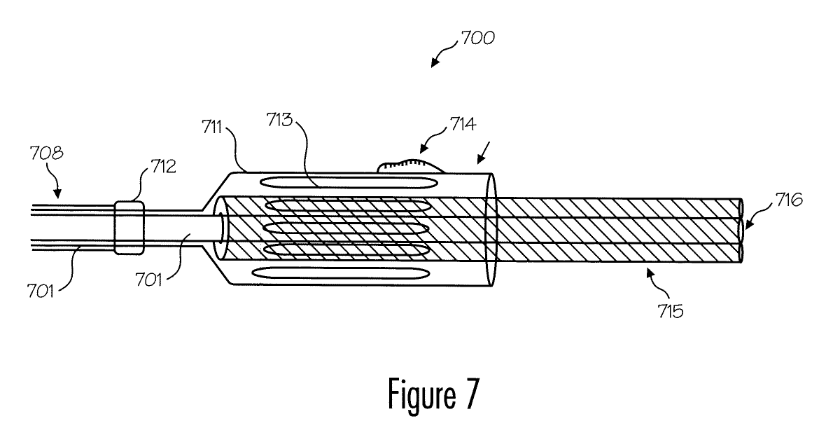

FIG. 7 shows a control mechanism for a dilation device according to one embodiment of the invention. Control mechanism is the hand-held portion of a dilation system (which is preferably a catheter that includes the controls and the device) and may be used with both spiraled and non-spiraled, expansive dilation devices. In the case of a non-spiraled, expansive dilation device, handle 711 is attached to outer sheath 708 through hemostatic value 712. For both spiraled and non-spiraled dilation devices, catheter 701 runs through handle 711 and has a wire port 716 at its proximal end. Handle 711 may include surface texturing 713 for easier grip. As shown in FIG. 7, handle 711 is a nut-type handle that is either fused to a secondary sheath and may be twisted (for a spiraled dilation device) or pushed/pulled (for a non-spiraled, expansive dilation device) to engage or disengage a dilation device. Handle 711 may also include a threaded, bolt-type fixation handle 715 that is fused to catheter 701. This allows for execution of a twisting motion for spiraled dilation devices. Handle 711 may also include a thumb-controlled quick release 714. Quick release 714 disengages handle 711 from the bolt-type fixation handle, allowing push/pull motions to be exerted on the handle and any attached sheaths and/or catheters (e.g., for engaging non-spiraled, expansive dilation devices).

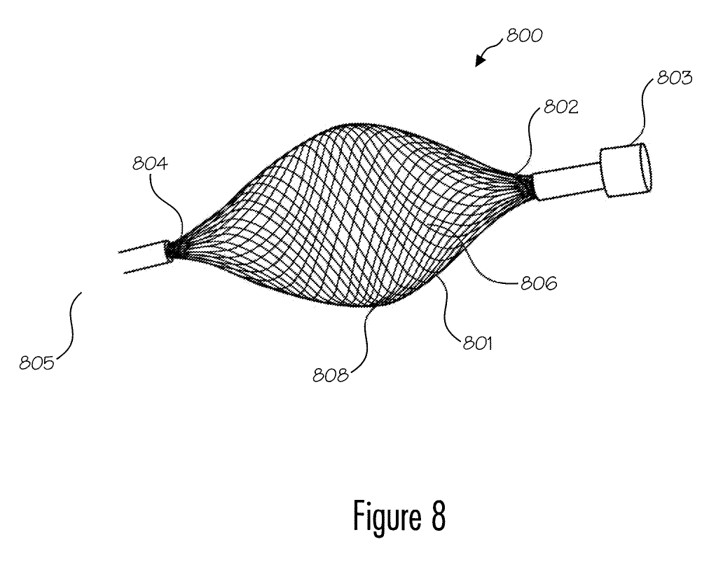

FIG. 8 shows an alternate device 800 according to the invention that is shown in a dilated position. Device 800 is comprised of wires 801 and includes a proximal end 802 retained by a retention member 803 and a distal end 804 retained by a retention member 805. As used herein, the distal end and the proximal end are the parts of the device that extend 15 mm from each respective retention member. Device 800 has a body portion 807 positioned between ends 802 and 804 and spaces 806 are formed between wires 801 when device 800 is dilated as shown. Spaces 806 are greater between wires 801 in body portion 807 than the spaces 806 between the wires 801 at end 802 or end 803 when device 800 is dilated. In this embodiment a band 808 of wires is formed near the center of body portion 807 to add greater radial strength, and hence the spaces between the wires 801 in band 808 are smaller than the spaces between the wires 801 in other parts of body portion 807.

FIG. 9 shows a device 900 according to the invention that is in the dilated position and comprises a plurality of wires 901. In this embodiment each wire 901 is parallel to the other wires 901 (in this context “parallel” means substantially parallel). Each of the wires 901 is also parallel to the vessel flow path when device 900 is inserted into a vessel (again, in this context, “parallel” means substantially parallel). Device 900 as shown is formed by slitting a tube and has unslitted ends 902 and 904 that are connected, respectively, to proximal end 906 and distal end 908. Device 900 has a body portion 910 between proximal end 906 and distal end 908. As shown, wires 901 are formed in three-wire groups with distances 912 between the groups and distances 914 between wires in each group. Distances 912 are greater than distances 914 and each of the respective distances 912 and 914 are greater in body portion 910 than they are at either proximal end 906 or distal end 908.

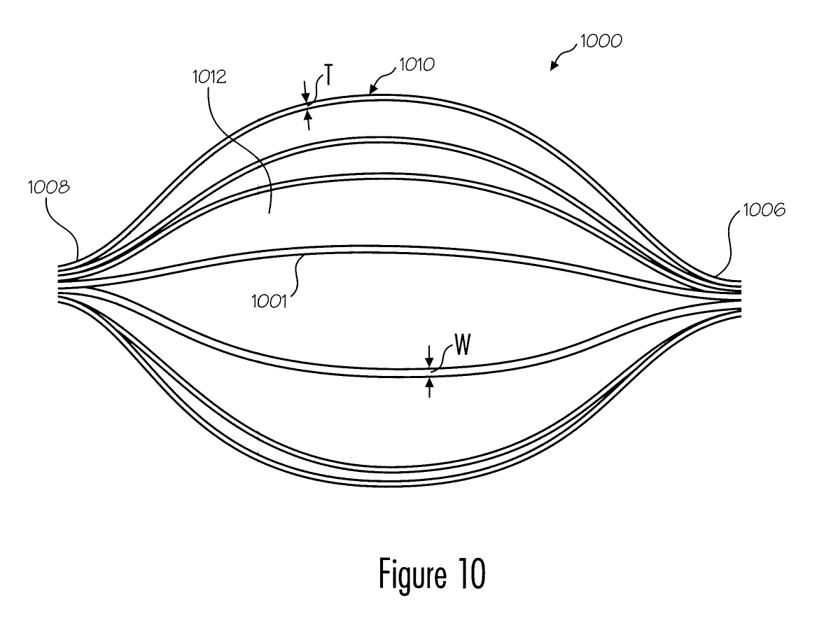

FIG. 10 shows a device 1000 that is in a dilated position. Device 1000 comprises a plurality of wires 1001 and is preferably formed by slitting a tube and leaving the ends of the tube (not shown in this Figure) unslit. In this embodiment each of the wires 1001 is parallel (in this context “parallel” means substantially parallel) to the other wires 1001 and each of the wires 1001 is also parallel (again, in this context, “parallel” means substantially parallel) to the vessel flow path when device 1000 is positioned in a vessel. Each wire 1001 is preferably a slat having a generally rectangular cross section and preferably having a width W greater than its thickness T. Width W could be any suitable width but is preferably between 0.020″ and 0.050″ and thickness T could be any suitable thickness but is preferably between 0.008″ and 0.015″. Device 1000 has a proximal end 1006, a distal end and 1008 and a body portion 1010. There is a distance 1012 between wires 1001 and in this embodiment the distance 1012 is greater in body portion 1010 that in either proximal end 1006 or distal end 1008.

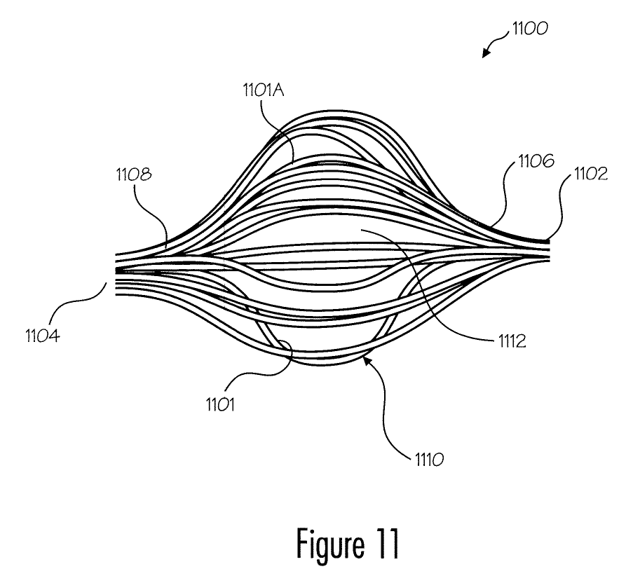

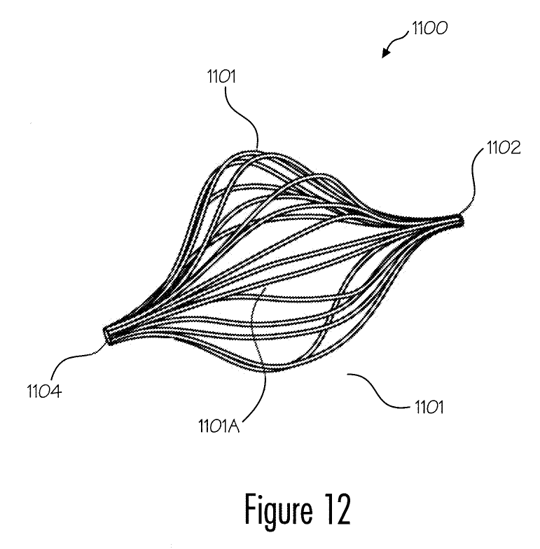

FIGS. 11 and 12 show a device 1100 according to the invention that is in a dilated position and that comprises a plurality of wires 1101. In this embodiment each wire 1101 is parallel to the other wires 1101 (in this context “parallel” means substantially parallel). Each of the wires 1101 are also parallel to the vessel flow path when device 1100 is inserted into a vessel (again, in this context, “parallel” means substantially parallel). Device 1100 as shown is formed by slitting a tube and has unslitted ends 1102 and 1104 that are connected, respectively, to proximal end 1106 and distal end 1108. Device 1100 has a body portion 1110 between proximal end 1106 and distal end 1108. Device 1100 has two types of wires, wires 1101 and 1101A. As shown wires 1101 are slender, having a preferred width of between about 0.008″ and 0.014″ whereas wires 1101A are wider and have a width of between about 0.020″ and 0.025.″ Wires 1101 also extend further from the center of body portion 1110 than do wires 1101A. In this embodiment wires 1101 and 1101A function together to apply even pressure to a substantial area of a vessel and/or apply even pressure to a substantial area of a structure to be positioned within a vessel.

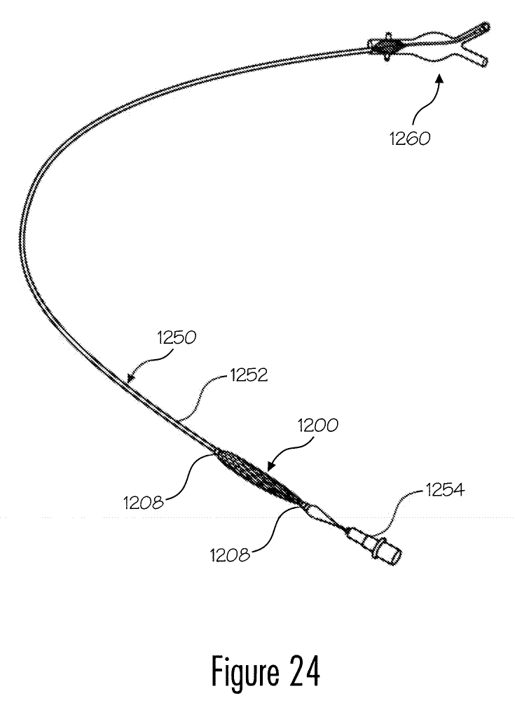

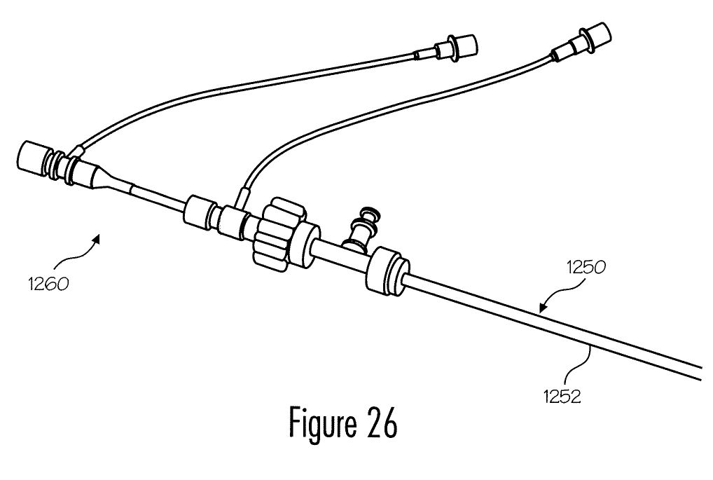

FIG. 13 shows a device 1200 according to the invention that is mounted on a catheter 1250. Catheter 1250 is of a design generally known in the art and includes an outer sheath 1252, a distal end 1260 (best seen in FIGS. 24 and 26) which is outside of the body portion and juxtaposed the operator when in use and a proximal end 1254 that is inserted into the body. Utilizing catheter 1250 a device, such as device 1200 or 1300, is dilated by pressing the distal and proximal ends of the device towards each other (or one end may be pressed towards the other while the other remains stationary) as discussed previously to some extent with respect to FIG. 6. Using this procedure a device is dilated by decreasing the distance between the proximal end and the distal end. The device is collapsed by releasing the force pushing the two ends together. Alternatively, any device according to the invention may be preformed in a dilated position and compressed into a dilated position when covered by catheter outer sheath 1252. When outer sheath 1252 is released the preformed device would immediately expand to its dilated position and then could be further dilated or collapsed by an operator utilizing the catheter.

When catheter 1250 and any device according to the invention, such as device 1200, that is mounted on catheter 1250 are inserted into a vessel, outer sheath 1252 would preferably at least partially cover the device to help retain it in its collapsed position and to allow for ease in directing the catheter and device through the vessel. Outer sheath 1252 is retracted to expose device 1200 when device 1200 is properly positioned in a vessel. If a device according to the invention were being used to position a structure in the vessel, the structure (such as a stent graft) could be mounted on the device in a typical manner known to those in the art so that as the device dilates the structure is dilated.

In FIG. 13, device 1200 is shown in its dilated position and it comprises a plurality of wires 1201 that are formed in a criss-cross pattern. Device 1200 has retention ends 1202 and 1204 that may be formed as part of catheter 1250, a distal end 1206, a distal end 1208 and a body portion 1210. Spaces 1212 are formed between wires 1201 and can be of any suitable size, e.g., between about 1 mm² and about 400 mm^{2,} as previously described. As shown, spaces 1212 are larger in body portion 1210 that in either proximal end 1206 or distal end 1208.

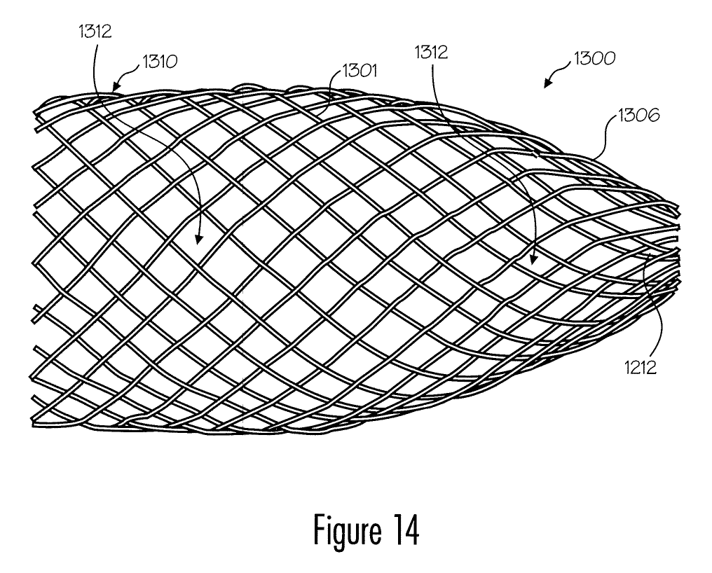

FIG. 14 is a close-up, partial side view of an alternate device 1300 showing proximal end 1306 and part of body portion 1310. As can be seen spaces 1312 between wires 1301 are smaller at proximal end 1306 than at body portion 1310.

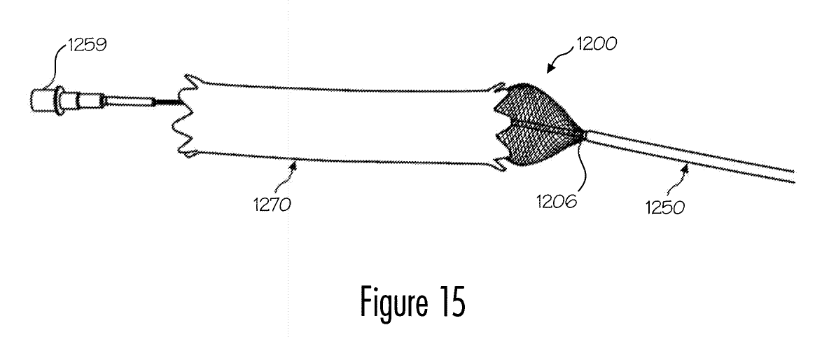

FIG. 15 illustrates how device 1200 can be utilized to dilate a stent graft 1270.

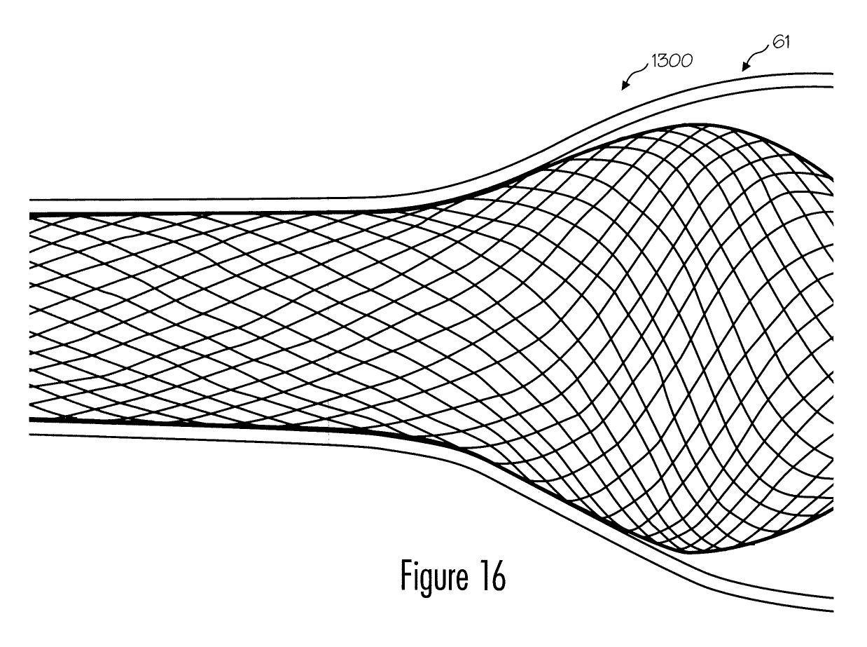

FIG. 16 shows device 1300 dilated in a plastic model G1 to simulate device 1200 conforming to a diameter disparity ratio of approximately 1.8:1 in a vessel.

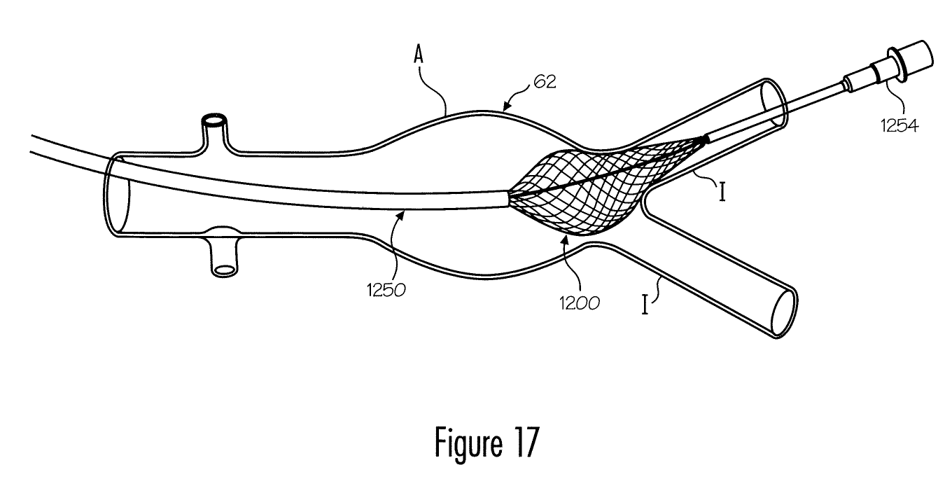

FIG. 17 shows device 1200 and catheter 17 in a plastic model G2 that simulates the aorta A and the iliac arteries I. In this Figure device 1200 is simultaneously positioned in the aorta and an iliac artery and is conforming to a multi-vessel diameter disparity ratio of about 2.0:1.

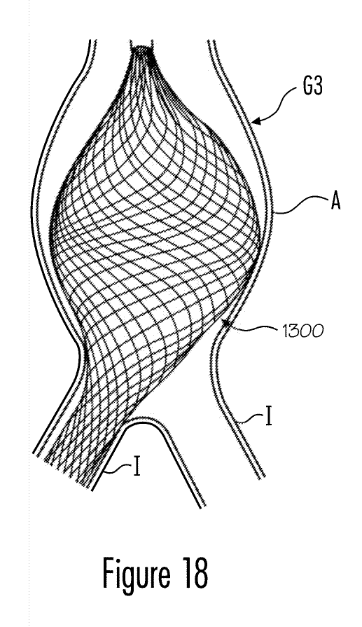

FIG. 18 shows a device 1300 in accordance with the invention that is dilated in a plastic model G3 to simulate device 1300 being dilated simultaneously in the aorta A and an iliac artery I. In this Figure device 1300 is conforming to a multi-vessel diameter disparity ratio of about 3.4:1.

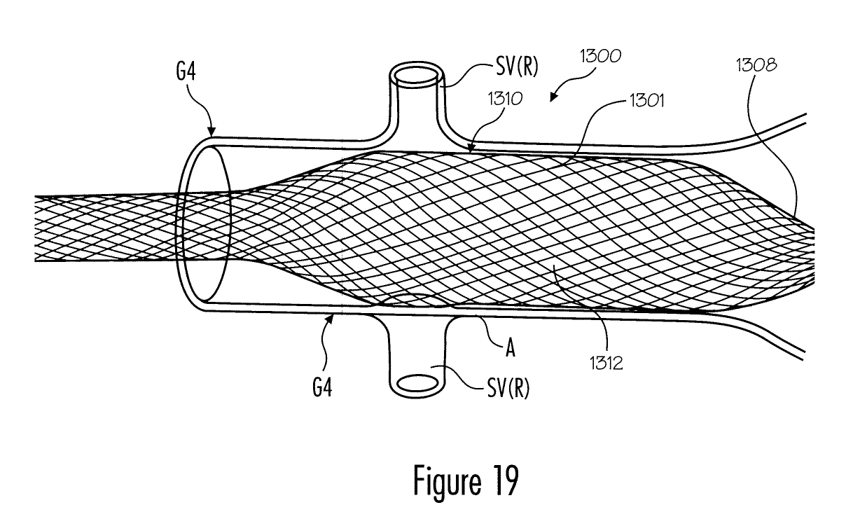

FIG. 19 shows device 1300 with wires 1301, proximal end 1308 and spaces 1312 between wires 1301. Device 1300 is dilated in a plastic model G4 to simulate device 1300 being dilated in aorta A and covering side vessels SV(R) that simulate the renal arteries. As can be seen, fluid would flow through the spaces 1312 at proximal end 1308, through the aorta and into the side vessels through spaces 1312 in body portion 1310. In this Figure, device 1300 is also conforming to a vessel diameter disparity ratio of about 2.0:1.

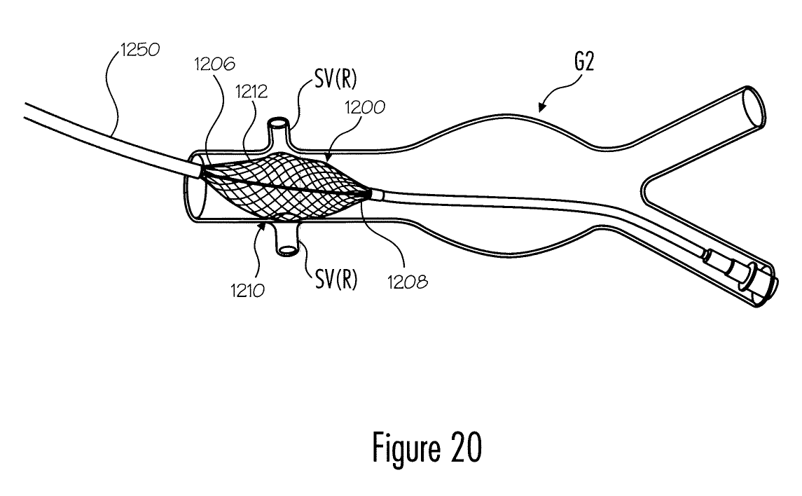

FIG. 20 shows device 1200 and catheter 1250 positioned in a plastic model G2 to simulate device 1200 being positioned and dilated in the aorta and covering side branches, such as the renal arteries SV(R). The spaces 1212 between the wires 1201 in device 1200 allow fluid to flow through the aorta and into the side vessels when device 1200 is dilated.

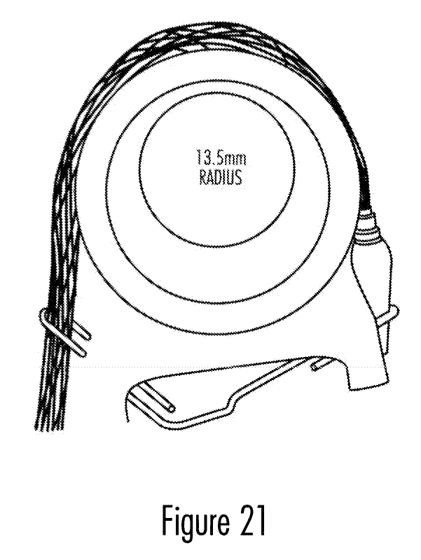

FIG. 21 shows the device of FIG. 13 in its collapsed position and having a kink radius of 13.5 mm.

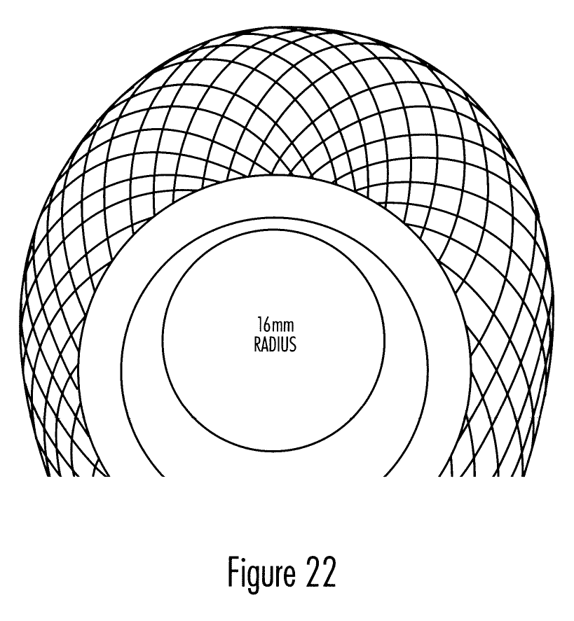

FIG. 22 shows the device of FIG. 13 in its fully dilated position and having a kink radius of 16 mm.

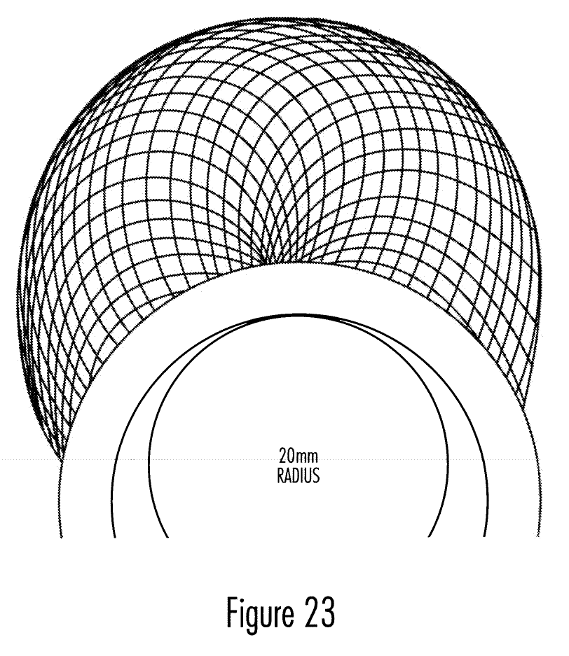

FIG. 23 shows the device of FIG. 13 in its fully dilated position and having a kink radius of 20 mm.

A device according to the present invention thus may have a kink radius of 13.5 mm or greater before being dilated. This includes kink radii of 14.0 mm, 15.0 mm, 16.0 mm, 17.0 mm, 18.0 mm, 19.0 mm, 20.0 mm and greater. Further, a device according to the present invention may, when fully dilated, have a kink radius of 16.0 mm. This includes kink radii of 17.0 mm, 18.0 mm, 19.0 mm, 200 mm, 21.0 mm, 22.0 mm, 23.0 mm, 24.0 mm, 25.0 mm and greater.

FIG. 24 shows the catheter 1250 of FIG. 13 that includes device 1200. Catheter 1250 has a proximal end 1254 that is inserted into a vessel during use, and a distal end 1260 that remains outside of the vessel and is used by an operator to position, release and dilate device 1200.

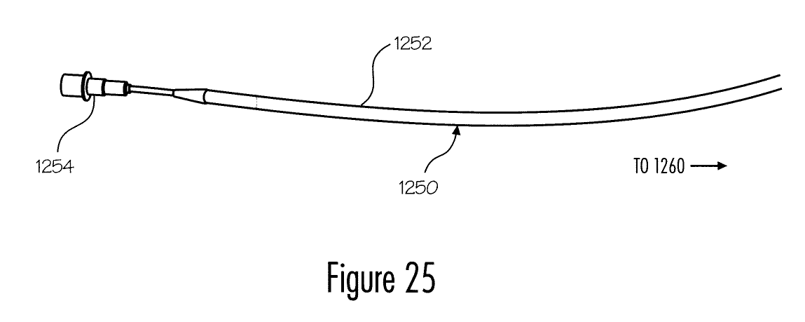

FIG. 25 shows proximal end 1254 of catheter 1250.

FIG. 26 shows distal end 1260 of catheter 1250.

Other embodiments of the invention will be apparent to those skilled in the art from consideration of the specification and embodiments disclosed herein. Thus, the specification and examples are exemplary only, with the true scope and spirit of the invention set forth in the following claims and legal equivalents thereof.

What is claimed:

1. A device for dilating a vessel or a structure positioned within a vessel, the device comprising a plurality of wires that are spaced apart when the device is dilated to allow for the passage of fluid through the device, the device being sufficiently compliant so that when placed into a vessel and dilated it can conform to at least one diameter disparity ratio of between 1.2:1 and 3.0:1.

2. The device of claim 1 that can conform to a diameter disparity ratio of 1.2:1.

3. The device of claim 1 that can conform to a diameter disparity ratio of 1.4:1.

4. The device of claim 1 that can conform to a diameter disparity ratio of 1.6:1.

5. The device of claim 1 that can conform to a diameter disparity ratio of 1.8:1.

6. The device of claim 1 that can conform to a diameter disparity ratio of 2.0:1.

7. The device of claim 1 that can conform to a diameter disparity ratio of 2.2:1.

8. The device of claim 1 that can conform to a diameter disparity ratio of 2.4:1.

9. The device of claim 1 that can conform to a diameter disparity ratio of 2.6:1.

10. The device of claim 1 that can conform to a diameter disparity ratio of 2.8:1.

11. The device of claim 1 that can conform to a diameter disparity ratio of 3.0:1.

12. The device of claim 1 wherein the wires are formed in a criss-cross pattern.

13. The device of claim 1 wherein each of the wires are parallel to the vessel flow path when in the vessel.

14. The device of claim 1 wherein at least some of the wires form a criss-cross pattern.

15. The device of claim 1 wherein the wires are comprised of nitinol.

16. The device of claim 1 wherein the wires have a circular cross-sectional and a diameter of between 0.008″ and 0.012″.

17. The device of claim 1 that further includes a permeable membrane on at least part of the device.

18. The device of claim 17 wherein the device has an outer surface and the permeable membrane is positioned on the outer surface.

19. A catheter including the device of claim 1 and comprising a sheath that at least partially encloses the device during insertion of the device into the vessel, the device being preshaped to automatically expand when released from the sheath.

20. A device for dilating two vessels or a structure positioned within two vessels, the device comprising a plurality of wires that are spaced apart when the device is dilated to allow for the passage of fluid through the device, the device being sufficiently compliant so that when it is simultaneously positioned in each of the two vessels it can conform to at least one multi-vessel diameter disparity ratio of between 1.2:1 and 3.0:1.

21. The device of claim 20 that can conform to a multi-vessel diameter disparity ratio of 1.2:1.

22. The device of claim 20 that can conform to a multi-vessel diameter disparity ratio of 1.4:1.

23. The device of claim 20 that can conform to a multi-vessel diameter disparity ratio of 1.6:1.

24. The device of claim 20 that can conform to a multi-vessel diameter disparity ratio of 1.8:1.

25. The device of claim 20 that can conform to a multi-vessel diameter disparity ratio of 2.0:1.

26. The device of claim 20 that can conform to a multi-vessel diameter disparity ratio of 2.2:1.

27. The device of claim 20 that can conform to a multi-vessel diameter disparity ratio of 2.4:1.

28. The device of claim 20 that can conform to a multi-vessel diameter disparity ratio of 2.6:1.

29. The device of claim 20 that can conform to a multi-vessel diameter disparity ratio of 2.8:1.

30. The device of claim 20 that can conform to a multi-vessel diameter disparity ratio of 3.0:1.

31. The device of claim 20 wherein each of the wires are parallel to the vessel flow path when in the vessel.

32. The device of claim 20 wherein at least some of the wires are formed in a criss-cross pattern.

33. A catheter including the device of claim 20 and comprising a sheath that at least partially encloses the device during insertion of the device into the vessel, the device being preshaped to automatically expand when released from the sheath.

34. The device of claim 20 wherein the wires are comprised of nitinol.

35. The device of claim 20 wherein the wires have a circular cross-section and a diameter of between 0.008″ and 0.012″.

36. The device of claim 20 that further includes a permeable membrane on at least part of the device.

37. The device of claim 36 wherein the device has an outer surface and the permeable membrane is positioned on the outer surface.

38. A device for dilating a vessel or a structure within a vessel, the device comprising a plurality of wires that are spaced apart when the device is dilated to allow for the passage of fluid through the device, the device comprising:
(a) a distal end;
(b) a proximal end; and
(c) a body portion between the distal end and the proximal end;
wherein at least some of the spaces between the wires in the body portion are larger than the spaces between the wires at the distal end or the proximal end when the device is dilated within a vessel, so as to allow fluid to flow into side vessels if the device is positioned against a side vessel.

39. The device of claim 38 wherein at least some of the spaces between the wires in the body portion have an area of no greater than 1 mm² when the device is dilated.

40. The device of claim 38 wherein at least some of the spaces between the wires in the body portion have an area of no greater than 5 mm² when the device is dilated.

41. The device of claim 38 wherein at least some of the spaces between the wires in the body portion have an area of no greater than 10 mm² when the device is dilated.

42. The device of claim 38 wherein at least some of the spaces between the wires in the body portion have an area of no greater than 25 mm² when the device is dilated.

43. The device of claim 38 wherein at least some of the spaces between the wires in the body portion have an area of no greater than 50 mm² when the device is dilated.

44. The device of claim 38 wherein at least some of the spaces between the wires in the body portion have an area of no greater than 100 mm² when the device is dilated.

45. The device of claim 38 wherein at least some of the spaces between the wires in the body portion have an area of no greater than 200 mm² when the device is dilated.

46. The device of claim 38 wherein the wires are comprised of nitinol.

47. The device of claim 38 wherein the wires have a circular cross-sectional and a diameter of between 0.008″ and 0.012″.

48. The device of claim 38 that further includes a permeable membrane on at least part of the device.

49. The device of claim 48 wherein the device has an outer surface and the permeable membrane is positioned on the outer surface.

50. The device of claim 37 wherein the body portion includes a band in which the size of the spaces between the wires in the band is less than the size of the spaces in the rest of the body portion.

51. A catheter including the device of claim 38 and comprising a sheath that at least partially encloses the device during insertion of the device into the vessel, the device being preshaped to automatically expand when released from the sheath.

52. A device for dilating a vessel or a structure within a vessel, the device comprising a plurality of wires that are spaced apart when the device is dilated to allow for the passage of fluid through the device, the wires being parallel to each other and being parallel to the vessel flow path when in the vessel.

53. The device of claim 52 that includes at least four spaced-apart wires.

54. The device of claim 52 that includes at least six spaced-apart wires.

55. The device of claim 52 that includes at least eight spaced-apart wires.

56. The device of claim 52 that includes at least ten spaced-apart wires.

57. The device of claim 52 that includes at least twelve spaced-apart wires.

58. The device of claim 52 that includes at least fourteen spaced-apart wires.

59. The device of claim 52 that includes at least sixteen spaced-apart wires.

60. The device of claim 52 that includes at least eighteen spaced-apart wires.

61. The device of claim 52 that includes at least twenty spaced-apart wires.

62. The device of claim 52 that includes at least twenty-four spaced-apart wires.

63. The device of claim 52 wherein the maximum space between each wire when the device is fully dilated is between 1 mm and 2 mm.

64. The device of claim 52 wherein the maximum space between each wire when the device is fully dilated is between 2 mm and 3 mm.

65. The device of claim 52 wherein the maximum space between each wire when the device is fully dilated is between 3 mm and 4 mm.

66. The device of claim 52 wherein the maximum space between each wire when the device is fully dilated is between 4 mm and 6 mm.

67. The device of claim 52 wherein the maximum space between each wire when the device is fully dilated is between 6 mm and 8 mm.

68. The device of claim 52 wherein the maximum space between each wire when the device is fully dilated is between 8 mm and 10 mm.

69. The device of claim 52 wherein the maximum space between each wire when the device is fully dilated is between 10 mm and 25 mm.

70. The device of claim 52 wherein the maximum space between each wire when the device is fully dilated is between 25 mm and 50 mm.

71. The device of claim 52 wherein the maximum space between each wire when the device is fully dilated is between 50 mm and 100 mm.

72. The device of claim 52 wherein each wire is a slat.

73. The device of claim 52 that further includes a permeable membrane on at least part of the device.

74. The device of claim 73 wherein the device has an outer surface and the permeable membrane is positioned on the outer surface.

75. A catheter including the device of claim 52 and comprising a sheath that at least partially encloses the device during insertion of the device into the vessel, the device being preshaped to automatically expand when released from the sheath.

76. A device for dilating a vessel or a structure within a vessel, the device comprising a plurality of wires that are spaced apart when the device is dilated to allow for the passage of fluid through the device, wherein the device before being dilated has a kink radius of greater than or equal to 13.5 mm.

77. The device of claim 76 wherein at least some of the wires are comprised of nitinol.

78. The device of claim 72 wherein the wires have a circular cross section and are between 0.008″ and 0.012″ in diameter.

79. The device of claim 72 wherein at lest some of the wires form a criss-cross pattern, the device has a body portion and the space between at least some of the wires in the body portion is between 2 mm² and 400 mm² when the device is fully dilated.

80. A device for dilating a vessel or a structure within a vessel, the device comprising a plurality of wires that are spaced apart when the device is dilated to allow for the passage of fluid through the device, wherein the device when fully dilated has kink radius of greater than or equal to 16 mm.

81. The device of claim 80 wherein the device has kink radius of greater than or equal to 20 mm when the device is fully dilated.

82. The device of claim 80 wherein the wires are comprised of nitinol.

83. The device of claim 80 wherein the wires have a circular cross section and are between 0.008″ and 0.012″ in diameter.

84. The device of claim 80 wherein at least some of the wires form a criss-cross pattern, the device has a body portion and the space between at least some of the wires in the body portion is between 2 mm² and 400 mm² when the device is fully dilated.

85. A device for dilating a vessel or structure within a vessel, the device comprising a plurality of wires that are spaced apart when the device is dilated to allow for the passage of fluid through the device, wherein when the device is positioned against a side vessel the pressure drop in the side vessel is less than 70% when the device is dilated, as measured in a pressure monitoring test utilizing water.

86. The device of claim 85 wherein the pressure drop is less than 60%.

87. The device of claim 85 wherein the pressure drop is less than 50%.

88. The device of claim 85 wherein the pressure drop is less than 40%.

89. The device of claim 85 wherein the pressure drop is less than 30%.

90. The device of claim 85 wherein the pressure drop is less than 20%.

91. The device of claim 85 wherein the pressure drop is less than 10%.

92. The device of claim 85 wherein the pressure drop is less than 5%.

93. The device of claim 85 wherein the pressure drop is less than 2%.

94. The device of claim 85 wherein the pressure drop is less than 1%.

95. A device for dilating a vessel or a structure within a vessel, the device comprising a plurality of wires that are spaced apart when the device is dilated to allow for the passage of blood through the device, the device capable of conforming to incongruent vessel shapes.

96. A device for dilating a vessel or structure within a vessel, the device comprising a plurality of wires that are spaced apart when the device is dilated to allow for the passage of fluid through the device, the device being sufficiently compliant so that when placed into a vessel it can conform to one or more diameter disparity ratios between 1.2:1 and 3.0:1, and can conform to one or more multi-vessel diameter disparity ratios between 1.2:1 and 3.0:1, wherein the device includes a distal end, a proximal end and a body portion between the distal end and the proximal end and spaces between the wires wherein at least some of the spaces between the wires in the body portion are larger than the spaces between the wires at the distal end or the proximal end when the device is dilated.

97. The device of claim 38 wherein at least some of the spaces between the wires in the body portion have an area of no greater than 400 mm² when the device is fully dilated.

98. The device of claim 1 that includes a proximal end and a distal end and that is dilated by decreasing the distance between the proximal end and the distal end.

99. The device of claim 20 that includes a proximal end and a distal end and that is dilated by decreasing the distance between the proximal end and the distal end.

100. The device of claim 38 that includes a proximal end and a distal end and that is dilated by decreasing the distance between the proximal end and the distal end.

101. The device of claim 52 that includes a proximal end and a distal end and that is dilated by decreasing the distance between the proximal end and the distal end.
